(19) United States
(12) Patent Application Publication
(10) Pub. No.: US 2014/0228880 A1
(43) Pub. Date: Aug. 14, 2014

(54) SOFT TISSUE-TO-BONE SUTURING AND MONITORING DEVICE

(71) Applicants: Leslie John Bisson, Williamsville, NY (US); John M. Marzo, Williamsville, NY (US)

(72) Inventors: Leslie John Bisson, Williamsville, NY (US); John M. Marzo, Williamsville, NY (US)

(21) Appl. No.: 14/177,856

(22) Filed: Feb. 11, 2014

Related U.S. Application Data
(60) Provisional application No. 61/763,216, filed on Feb. 11, 2013.

(51) Int. Cl. A61B 17/04

(52) U.S. Cl. CPC A61B 17/0401
USPC 606/232

(57) ABSTRACT

A surgical implant for monitoring stress loads applied to a sutured soft tissue-to-bone repair including a securing device containing sutures to effect a soft tissue-to-bone repair and a monitoring means whereas the loads applied to the sutures are detected by a load sensor powered by a local source and sent by a transmitter to a receiver. The load sensor, power source and transmitter are all arranged within the securing device. The monitoring means is arranged to transmit at least one input to a receiver.

### CROSS-REFERENCE TO RELATED APPLICATIONS

This patent application claims the benefit under 35 U.S.C. §119(e) of U.S. Provisional Patent Application No. 61/763,216, filed Feb. 11, 2013, which application is incorporated herein by reference.

### FIELD OF THE INVENTION

The invention broadly relates to a device for monitoring tension loads applied to a sutured soft tissue-to-bone repair. More specifically, the invention relates to a surgically implanted device for monitoring tension loads applied to a sutured soft tissue-to-bone repair which transmits the measured loads, for example wirelessly, so that the healing of the tissue-to-bone repair can be monitored.

### BACKGROUND OF THE INVENTION

Soft tissues such as muscles, ligaments and tendons throughout the human body can be damaged for example, by a sprain, strain, a contusion or overuse of a particular part of the body. Such injuries can result in pain, swelling, bruising and loss of function. Rotator cuff tears are tears of one or more of the four tendons of the rotator cuff muscles. In order to repair such soft tissue tear injuries surgery is sometimes required. Surgical repair of a rotator cuff tear involves reattaching the tendon to bone. Small rivets, suture anchors, or the like, are used to attach the tendon to the bone. Full recovery after rotator cuff surgery often takes at least 4 to 6 months and involves a variety of therapeutic stages including passive motion, active motion, strengthening and full activity. Full recovery time varies depending on a number of factors such as systemic diseases e.g., CVD, diabetes mellitus, etc., behavioral factors e.g., smoking and obesity, age, gender, and degree of degeneration of the repaired tissue. Moreover, the size of the tear, the chronicity, the amount of muscle atrophy and the degree of tendon retraction affect recovery time. As the tear heals, the amount of load-sharing between a repair device or construct, i.e., suture, anchors, screws, and the repaired tissue lessens as the repaired tissue becomes dominant.

It is known that individuals with differences in the above factors, as well as other unlisted factors, will heal at different rates, and should be rehabilitated differently following soft-tissue repair surgery. There is a progressive change in the amount of load seen on any repair device or construct with time, including suture, anchors, screws, etc., as healing takes place and the strength of the repair tissue becomes the dominant factor. The degree of load-sharing between repair construct and repair tissue varies due to the foregoing factors, and cannot be predicted accurately for any given individual. There is a large variation in success of soft-tissue repairs, with failure rates ranging from 1-2% for biceps tendon repairs to 5% for Achilles tendon repairs up to 50-75% for large, retracted rotator cuff tears.

Even repairs which have a low re-tear rate have a relatively high early post-operative morbidity, with length of time immobilized and activity limitations based on animal basic science studies of healing and clinical experience, rather than being based on individual rates of healing.

A person recovering from soft tissue-to-bone surgery, while rehabilitating the repaired tissue to function as undamaged tissue, must continually monitor the amount of load or stress on the repaired tissue to avoid re-injuring the tissue. Typically, this monitoring requires close supervision by a qualified individual such as a therapist. Since this healing process is monitored by humans the process is prone to error. The recommended exercises, the type and time of immobilization, and the permitted activities vary significantly from patient to patient.

U.S. Pat. No. 8,176,922 (Sherman et al.) discloses a knee implant system which post-operatively measures pressure in the knee joint after a total knee replacement and communicates data from a position in the body to an external data gathering device. However, the foregoing system only measures compressive forces between the tibial and femoral components of the knee.

U.S. Pat. No. 8,146,422 (Stein) discloses an artificial joint implant that measures loads at various points throughout the range of motion of the joint. The implant disclosed includes a sensor and wireless communication and is placed against or engaged with load-bearing surfaces.

U.S. Pat. No. 7,794,499 (Navarro et al.) discloses an artificial intervertebral spinal disc which responds to tensional forces between vertebrae. The artificial intervertebral spinal disc disclosed includes an upper plate and a lower plate separated by an elastic layer. As tensional forces are applied to the plates, end portions of restraining members activate sensors which provide a signal indicating that the restraining members have restrained the plates from exceeding their maximum allowable distances. Additionally, end portions provide a signal to prevent complete compression. The output signals are generated by a communications link and an antenna which create a wireless communications link between the electronics and an external processing functionality. However, the device disclosed provides no quantitative measurements of strain.

Direct, real-time information about the loads present at the tendon-bone repair site interface would allow accurate, individualized rehabilitation following surgery, with information about specific rehabilitation exercises, need for and length of immobilization, and activities permitted during the first several months following surgery being continuously available to the patient, therapist, and surgeon. The recommended exercises, the type and time of immobilization, and the activities permitted would vary significantly from patient to patient, and could be accurately monitored during the healing phase following surgery. The availability of this information to the patient would allow them to progress in their recovery following surgery in an individualized fashion, and would keep them from needlessly worrying about whether they have “overstressed” their repair at any point after surgery. After a series of laboratory and clinical studies, a database could establish “safe zones” for individual repairs that would be useful for treating patients without the implantable device.

Thus, there is a longfelt need for an electronic means for monitoring tension loads on a soft tissue-to-bone repair. Additionally, there is a longfelt need for an implant that continuously measures tension loads on a soft tissue-to-bone repair where such measurements are quantitative so that a patient's prognosis and rehabilitation can be individualized and effectively monitored, particularly in real time.

### BRIEF SUMMARY OF THE INVENTION

Broadly, the subject invention is a device which is surgically implanted into a subject and arranged to detect and record stress loads applied to a sutured soft tissue-to-bone repair. In an embodiment, the device is adapted to be implanted in the shoulder of a subject with a rotator cuff tendon sutured to bone; however, the device may be used to monitor tensional stress in any soft tissue-to-bone repair. The device includes a load sensor which is coupled to the sutures and/or directly to the suture anchor and measures the stress applied to the repair site over time and as the subject moves the portion of his body associated with the repaired soft tissue. The sensor is also coupled to a power source and a wired or wireless transmitter arranged to transmit the measured stress patterns to an external receiver. The external receiver may include a display means.

In one embodiment, the invention comprises a suture anchor which incorporates a miniaturized load sensor coupled to a power source and wireless transmitter which transmits continuous information about the loads occurring at the suture. These loads will vary according to the repair method as well as the factors listed above, and can be monitored by the patient, therapist, and surgeon, and can be used to guide activities in an individualized fashion following surgery. The invention may comprise an independent monitoring device which is placed across the repair at the completion of the surgery. The invention may comprise a monitoring device which is placed on top of the tendon, threaded onto the sutures from the suture anchor, which both acts to compress the tendon at the site of repair as well as monitor the loads on the device. This information is transmitted to a wireless transmitter which sends the load information to an external receiver. This receiver can be worn by the patient, or alternatively, can be used by the surgeon or therapist.

In one embodiment of the invention, the load sensor, transmitter, and power source are fixed to an implant, e.g., press fit, screwed in or other deployed fixation mechanism, embedded in the bone at a site near the repair site, and the load sensor is connected to the soft tissue by means of sutures utilized for the repair.

In another embodiment, the transmitter and power source are fixed to an implant, e.g., press fit, screwed in or other deployed fixation mechanism, embedded in the bone at a site near the repair site, and the load sensor is incorporated into the suture anchor and connected to the sutures used in the repair.

In yet another embodiment, the load sensor, transmitter, and power source may be arranged within a suture anchor or, alternatively, the load sensor may be arranged in a suture anchor and the power source and wireless transmitter may be fixed to a separate embedded screw. In either case, the load sensor measures the loads on the repair sutures. The load sensor may comprise a pulley coupled to the eyelet and to a strain gauge and digital voltage recorder, and possibly a wireless transmitter arranged to transmit the recorded loads to an external receiver, which may include a signal amplifier and digital output for a user.

A primary object of the present invention is to provide direct, real-time information about the tensile loads present at a soft tissue-to-bone repair-site.

Another object of the present invention is to provide accurate, individualized rehabilitation following a soft tissue-to-bone repair surgery, with information about specific rehabilitation exercises, need for and length of immobilization, and activities permitted during the first several months following surgery being continuously available to the patient, therapist, and surgeon.

These and other objects and advantages of the present invention will be readily appreciable from the following description of preferred embodiments of the invention and from the accompanying drawings and claims.

### BRIEF DESCRIPTION OF THE DRAWINGS

The nature and mode of operation of the present invention will now be more fully described in the following detailed description of the invention taken with the accompanying drawing figures, in which:

FIG. 1A is a perspective view of an embodiment of the present invention;

FIG. 1B is a top plan view of the present invention shown in FIG. 1A;

FIG. 1C is a cross sectional view of the embodiment of the present invention taken generally along line 1C-1C in FIG. 1B with the inner core exploded upwardly to better illustrate the invention;

FIG. 2A is a side elevational view of an embodiment of the present invention shown secured within a humerus bone of a human and around a rotator cuff of a human;

FIG. 2B is an enlarged side elevational view of encircled region 2B shown in FIG. 2A having all electronic components within the device;

FIG. 3A is a side elevational view of another embodiment of the present invention soft tissue-to-bone repair device;

FIG. 3B is a side elevational view of yet another embodiment of the present invention soft tissue-to-bone repair device;

FIG. 4 is a schematic diagram of another embodiment of the present invention soft tissue-to-bone repair device including an external receiver, amplifier and digital output; and,

FIG. 5 is a side view of another embodiment of the present invention.

### DETAILED DESCRIPTION OF THE INVENTION

At the outset, it should be appreciated that like drawing numbers on different drawing views identify identical, or functionally similar, structural elements of the invention. While the present invention is described with respect to what is presently considered to be the preferred aspects, it is to be understood that the invention as claimed is not limited to the disclosed aspects.

Furthermore, it is understood that this invention is not limited to the particular methodology, materials and modifications described and as such may, of course, vary. It is also understood that the terminology used herein is for the purpose of describing particular aspects only, and is not intended to limit the scope of the present invention, which is limited only by the appended claims.

Unless defined otherwise, all technical and scientific terms used herein have the same meaning as commonly understood to one of ordinary skill in the art to which this invention belongs. Although any methods, devices or materials similar or equivalent to those described herein can be used in the practice or testing of the invention, the preferred methods, devices, and materials are now described.

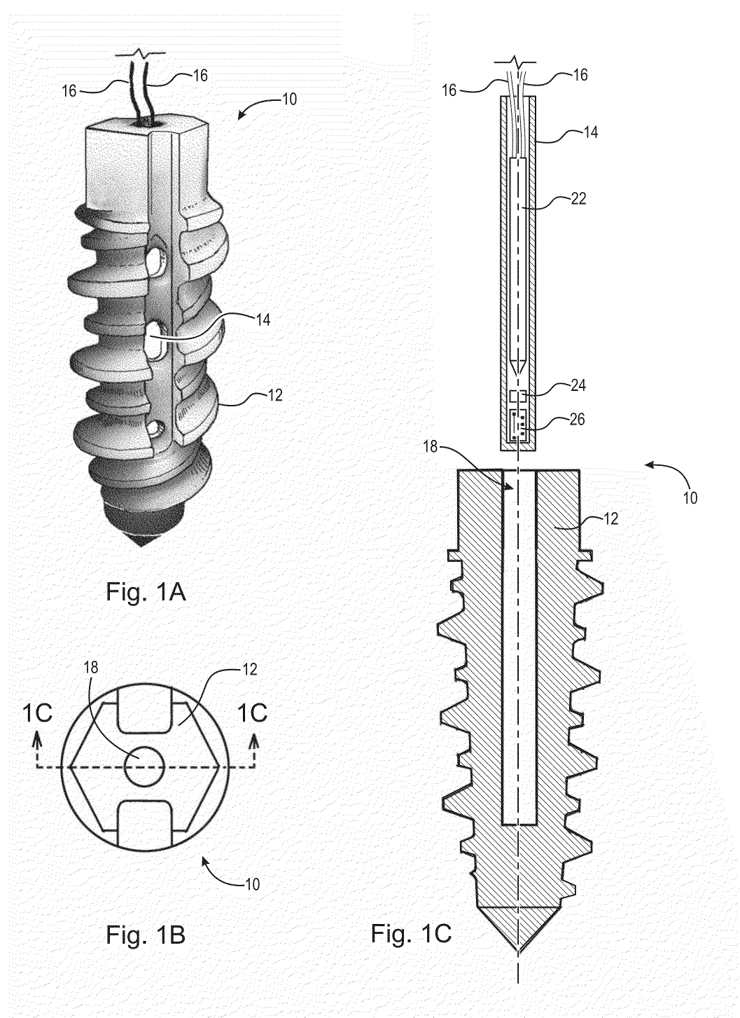

Adverting now to the figures, FIGS. 1A-1C show soft tissue-to-bone suturing and monitoring device 10 (hereinafter repair device 10). Repair device 10 broadly includes outer core 12, inner core 14 arranged within outer core 12. Inner core 14 comprises components to monitor the load-sharing between repair device 10 and the repaired soft tissue. In an embodiment, outer core 12 is a suture anchor but, it should be appreciated that outer core 12 could be any device which can be used to fix tendons and ligaments, i.e., soft tissue, to bone. It is well known in the art to use suture anchors for rotator cuff repairs. Outer core 12 is preferably a screw mechanism, but it could also be retained within the bone by an interference fit, press fit or deployable fixture. Additionally, outer core 12 can be made of metal, polymer or biodegradable material. Repair device 10 further includes suture 16 which is arranged to attach to outer core 12, inner core 14, and/or load sensor 22. Suture 16 can be made of non-absorbable material, e.g., a braided synthetic such as Ethibond, Orthocord or Fiberwire (Arthrex), or absorbable material, e.g., braided synthetic Polyglactin, coated braided synthetic Polyglactin with calcium stearate, or mono-filament synthetic Polydioxalone.

As is best understood in view of FIG. 1C, outer core 12 comprises bore 18 wherein inner core 14 is disposed. In an embodiment, inner core 14 is secured within outer core 12 by any means known in the art, e.g., back filling bore 18 with an epoxy resin after installing inner core 14. It should be appreciated that such securing must permit the transmission of force from sutures 16 to a load sensor, as discussed infra.

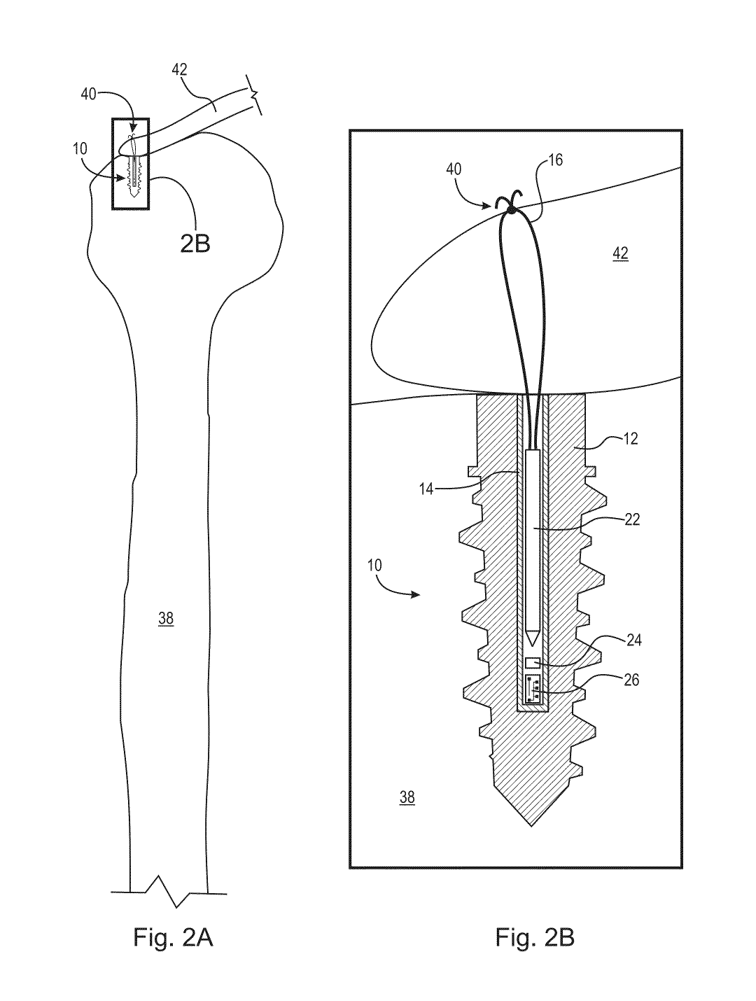

FIGS. 2A-2B depict a partial cross-section view of an embodiment of the present invention including suture 16, load sensor 22, battery 24 and transmitter 26 all arranged within inner core 14 of repair device 10. As the soft tissue heals, the soft tissue bears an increasing portion of the load and repair device 10 bears a decreasing portion of the load.

Load sensor 22 is arranged to communicate the amount of force or load exerted on sutures 16 of repair device 10. Load sensor 22 is preferably a strain gauge and a digital voltage recorder which monitors biological parameters such as strain and tension on sutures 16. However, it should be appreciated that load sensor 22 could be another type of sensor, e.g., piezoelectric sensor, and such variations are within the spirit and scope of the claimed invention. Battery 24 is the preferable power source used to power load sensor 22 and transmitter 26. Batteries suitable for use within the human body are well known in the art. Transmitter 26 is preferably arranged within inner core 14 and transmitter 26 is electrically coupled to load sensor 22. Transmitter 26 communicates, for example wirelessly, stress load measurements from load sensor 22 to external device or receiver 30. Such communication between transmitter 26 and external device or receiver 30 is well known in the art and may be achieved by radiofrequency or Bluetooth technology, for example. Receiver 30 may be worn by the patient, or alternatively, may be used by the surgeon or therapist. In some embodiments, receiver 30 is connected to amplifier 32 which increases the signal strength of the signal transmitted from inner core 14 to receiver 30. In some embodiments, amplifier 32 is connected to digital output 34 for further transmission of the signal received from inner core 14. In some embodiments, inner core 14 further comprises storage device 36. Storage device 36 may be any suitable storage means known in the art, e.g., random access memory, compact flash, eeprom, etc. In some embodiments, e.g., the embodiment depicted in FIG. 5, repair device 10 may include pulley 20 arranged within inner core 14 to facilitate longitudinal movement of suture 16 within repair device 10. Pulley 20 is preferably cylindrical and includes a groove for receiving suture 16. Pulley 20 further includes a securement device to secure pulley 20 to inner core 14. The preferred securement device includes a member extending from the cylindrical body of pulley 20 to the inside walls of inner core 14 such that pulley 20 rotates about the member, e.g., pin 28. It should be appreciated that including pulley 20 within inner core 14 is an optional feature and the present invention will provide real-time tensile load data with and without pulley 20 being included.

During a surgical repair of soft tissue-to-bone, repair device 10 is anchored to bone 38 and suture 16 is passed through tendon 42 and secured with knot 40. Suture 16 extends upwardly and outwardly from repair device 10, with suture 16 being passed through tendon 42 and tied with knot 40, effecting repair of tendon 42 to bone 38. In FIG. 2A, the tear is depicted as rotator cuff 42. Suture 16 is secured to rotator cuff 42 by suture 16 which passes through rotator cuff 42 and is tied in a knot at the outer surface of rotator cuff 42 (better shown in FIG. 2B). In this embodiment, sutures 16 act to both compress the soft tissue at the site of repair as well as monitor the loads on repair device 10.

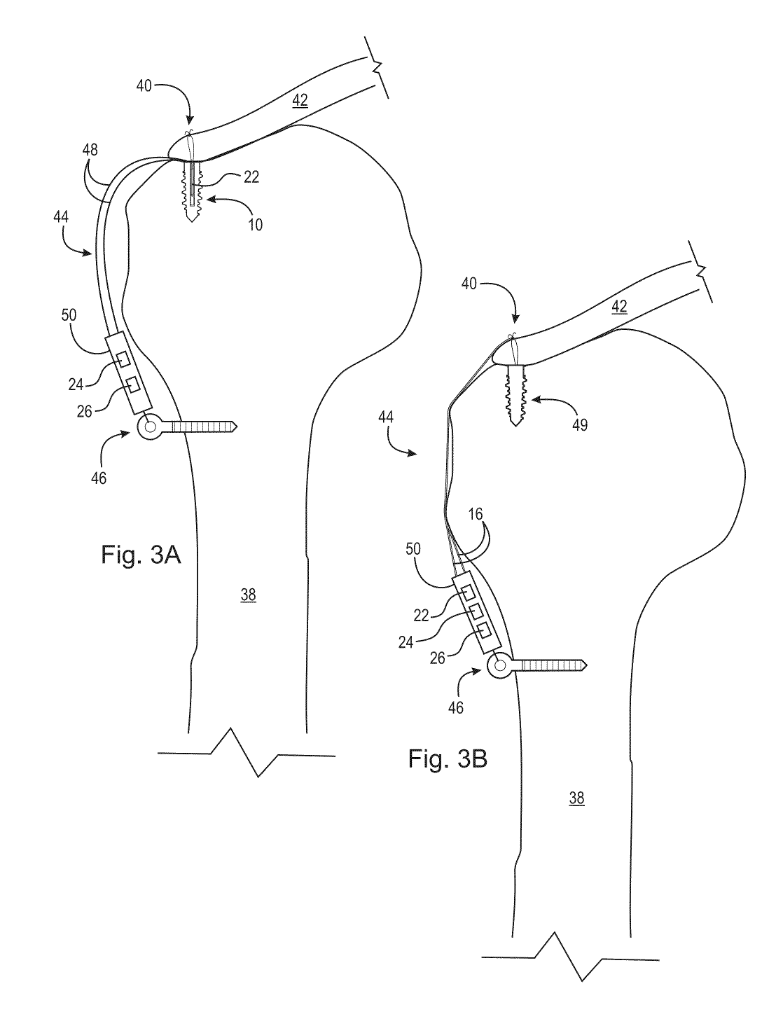

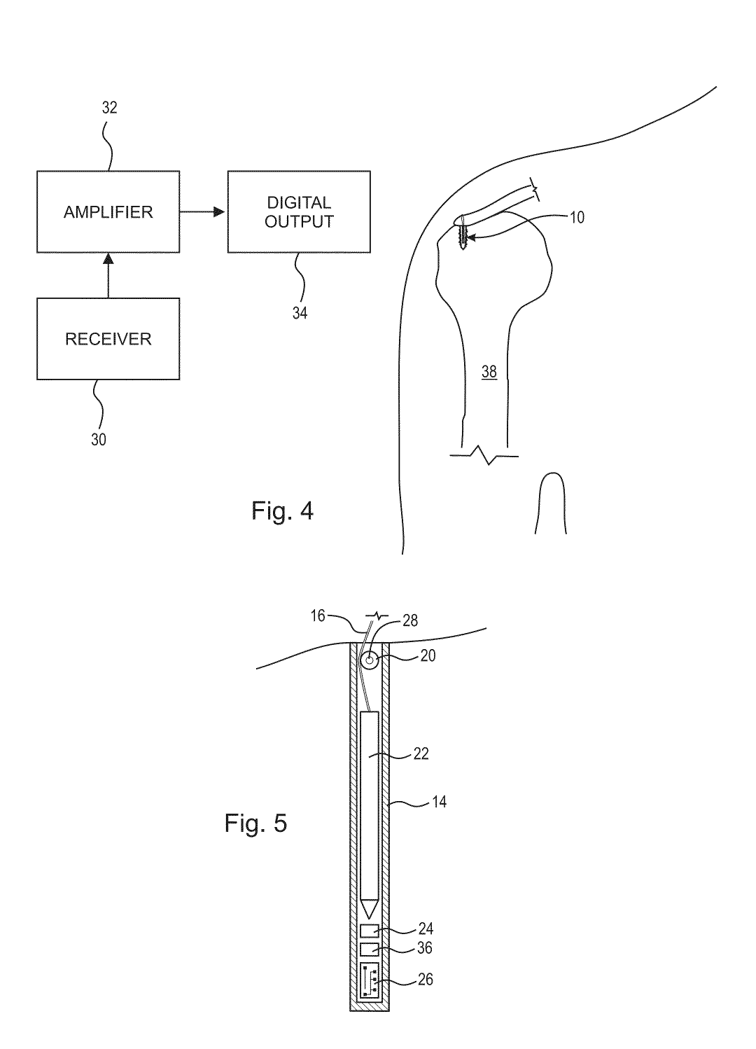

An alternate embodiment of the present invention is shown in FIG. 3A where battery 24 and transmitter 26 are removed from repair device 10 and arranged proximate attachment device 46. Repair device 10 includes the same structural elements as described above with respect to the embodiment shown in FIGS. 2A and 2B; however, inner core 14 only includes load sensor 22. Suture 16 is secured from repair device 10 to rotator cuff 42 as described above, while wires 48 pass data and power between load sensor 22, battery 24 and transmitter 26. In both the prior embodiment and the alternate embodiment shown in FIG. 3A, attachment device 46 is a typical surgical screw having an eyelet or opening proximate the head of the screw so that external component portion 50 can be secured to attachment device 46.

Another alternate embodiment of the present invention is shown in FIG. 3B where load sensor 22 is placed with transmitter 26 and battery 24 proximate attachment device 46 within external component portion 50. In this embodiment, suture 16 is secured from attachment device 49, to rotator cuff 42 and back to attachment device 46 via external component portion 50 which includes load sensor 22. It should be appreciated that although external component portion 50 is depicted as a separate element than attachment device 46, attachment device 46 may also be configured similarly to repair device 10, i.e., external component portion 50 may be positioned within attachment device 46, and such variations are within the spirit and scope of the invention.

It should be appreciated that the present invention may be used to measure threes in tendons and ligaments in vivo, e.g., daily activity, sports performance, rehabilitation, etc. Additionally, it may be used to assess strength of surgical repairs, e.g., during activities of daily living, and during sports to determine return to play, to guide rehabilitation, etc. Rehabilitation may be improved by using the present invention to design post-operative programs and controlling rehabilitation activities in real time. The present invention may be used in a variety of surgical repairs, including but not limited to, rotator cuff, achilles tendon, quadriceps tendon, patella tendon and biceps tendon repairs, as well as ACL reconstructions. Moreover, the present invention may also be used for surgical repair of medial collateral ligaments, lateral collateral ligaments, tricep tendons, glenohumeral ligaments, and a variety of other soft tissue to bone repair.

Thus, it is seen that the objects of the present invention are efficiently obtained, although modifications and changes to the invention should be readily apparent to those having ordinary skill in the art, which modifications are intended to be within the spirit and scope of the invention as claimed. It also is understood that the foregoing description is illustrative of the present invention and should not be considered as limiting. Therefore, other embodiments of the present invention are possible without departing from the spirit and scope of the present invention.

What is claimed is:

1. A surgical implant for monitoring stress loads applied to a sutured soft tissue-to-bone repair site, comprising:
an attachment device adapted for a soft tissue-to-bone repair;
a monitoring apparatus comprising a load sensor, a power source, and a transmitter, fixedly secured to the attachment device; and,
a suture loop adapted to affect the soft tissue-to-bone repair and to transmit a force between the soft tissue-to-repair site and the monitoring apparatus.

2. The surgical implant of claim 1 wherein the attachment device comprises an outer core formed from a metal, a polymer, a biodegradable material or combinations thereof.

3. The surgical implant of claim 1 wherein the attachment device comprises a screw is mechanism.

4. The surgical implant of claim 1 wherein the attachment device comprises an outer core having a bore disposed therein, an inner core comprising the monitoring device, and the inner core is disposed within the bore.

5. The surgical implant of claim 1 wherein the load sensor comprises at least one of: a strain gauge, a digital voltage recorder and a piezoelectric sensor.

6. The surgical implant of claim 1 wherein the monitoring apparatus further comprises a storage device.

7. The surgical implant of claim 1 further comprising a pulley arranged to transmit the force in a longitudinal direction.

8. The surgical implant of claim 11 wherein the force is proportional to the stress loads.

9. The surgical implant of claim 1 wherein the soft tissue-to-bone repair is selected from the group consisting of: a rotator cuff, an achilles tendon, a quadricep tendon, a patella tendon, a bicep tendon, an anterior cruciate ligament, a medial collateral ligament, a lateral collateral ligament, a tricep tendon and a glenohumeral ligament.

10. A surgical implant system for monitoring stress loads applied to a sutured soft tissue-to-bone repair site comprising the surgical implant of claim 1 and further comprising an external receiver adapted to receive real-time measurements of the force present at the sutured soft tissue-to-bone repair site.

11. The surgical implant system of claim 10 further comprising at least one of: an amplifier; and, a digital output.

12. A surgical implant for monitoring stress loads applied to a sutured soft tissue-to-bone repair site, comprising:
a first attachment device adapted for a soft tissue-to-bone repair;
a load sensor fixedly secured to a first attachment device;
a suture loop adapted to affect the soft tissue-to-bone repair and to transmit a force between the soft tissue-to-bone repair site and the load sensor;
a second attachment device arranged proximal to the soft tissue-to-bone repair site; and,
a power source and a transmitter fixedly secured to the second attachment device, wherein the load sensor is connected to the power source and the transmitter.

13. The surgical implant of claim 12 wherein the load sensor is connected to the power source and the transmitter by at least one wire.

14. A surgical implant for monitoring stress loads applied to a sutured soft tissue-to-bone repair, comprising:
a first attachment device adapted for a soft tissue-to-bone repair;
a first suture loop adapted to affect the soft tissue-to-bone repair;
a second attachment device arranged proximal to the soft tissue-to-bone repair site;
a monitoring apparatus comprising a load sensor, a power source, and a transmitter, fixedly secured to the second attachment device; and,
a second suture loop adapted to transmit a force between the soft tissue-to-repair site and the monitoring apparatus.
